(19) United States
(12) Patent Application Publication
(10) Pub. No.: US 2006/0062770 A1
(43) Pub. Date: Mar. 23, 2006

(54) Organogenesis from dissociated cells

(75) Inventors: Ying Zheng, West Chester, PA (US); Xiaobing Du, Philadelphia, PA (US); Wei Wang, Paoli, PA (US); Marylene Boucher, Philadelphia, PA (US); Satish Parimoo, Bridgewater, NJ (US); Kurt Stricker Stenn, Princeton, NJ (US)

Correspondence Address: MICHAEL BEST & FRIEDRICH, LLP, ONE SOUTH PINCKNEY STREET, P O BOX 1806, MADISON, WI, 53701, US

(73) Assignee: Aderans Research Institute, Inc., Beverly Hills, CA (US)

(21) Appl. No.: 11/203,804

(22) Filed: Aug. 15, 2005

Related U.S. Application Data
(60) Provisional application No. 60/601,496, filed on Aug. 13, 2004.

(51) Int. Cl. A61K 35/36

(52) U.S. Cl. 424/93.7

(57) ABSTRACT

A method assay for rapidly and reproducibly generating hair follicles from dissociated epithelial and mesenchymal cells is disclosed. The method serves both as a tool for measuring the trichogenic (i.e., hair growth-inducing) property of cells and for studying the mechanisms dissociated cells employ to assemble an organ. In a method of this application dissociated cells, isolated from newborn mouse skin, are injected into adult mouse truncal skin, hair follicles develop. This process involves the aggregation of epithelial cells to form clusters which are sculpted by apoptosis to generate “infundibular cysts”. From the “infundibular cysts” hair germs form followed by follicular buds and then pegs which grow asymmetrically to differentiate into cycling mature pilosebaceous structures. Using various techniques, exposure of the “infundibular cysts” by puncturing, piercing, or scratching the skin and, in an approach, covering the exposed cysts with a wound dressing material produced egressing hair follicles.

### CROSS-REFERENCE TO RELATED APPLICATIONS

This application claims priority to provisional patent application Ser. No. 60/601,496, filed Aug. 13, 2004, which application is incorporated in its entirety herein.

### BACKGROUND OF THE INVENTION

The goal of current bioengineering efforts is to generate or reconstitute fully organized and functional organ systems starting from dissociated cells that have been propagated under defined tissue culture conditions.

It has long been recognized that the hair follicle has profound regenerative ability, in that it cycles over the life-time of the individual and reproduces its lower half, in a Promethean manner, cycle after cycle (Stenn & Paus, 2001 and references therein). In fact, the hair follicle is one of the few biologic structures that continue to reform itself throughout the lifetime of the individual. The important question regarding this regeneration—as is the question in all regenerative systems—is how reformation of this organ occurs: by means of what cell interactions and what molecular messages and signals. Impetus to study the regenerative properties of the follicle have been stimulated by recent findings showing that 1) the follicle contains epithelial (Cotsarelis et al. 1990, Morris et al. 2004) and mesenchymal cell populations with stem cell properties (Jahoda et al. 2003); 2) follicle-derived cells can orchestrate the regeneration of the complete skin organ (Prouty et al. 1996, 1997) and appear to play a role in wound repair (Gharzi et al. 2003; Jahoda and Reynolds 2001); and 3) follicle derived cell populations can generate adipocytes, bone, cartilage and bone marrow on the one hand (Lako et al. 2002, Jahoda et al. 2003) and sebaceous glands, follicles and epidermis, on the other (Oshima et al. 2001; Taylor et al. 2000). The current paradigmatic model for hair follicle growth induction was ushered in with the demonstration that label-retaining cells rest within the bulge region of the follicle (Cotsarelis et al. 1990). By the bulge activation hypothesis, signals are delivered to the resting epithelial follicle from the papilla which then induces the next follicle cycle. Direct evidence that cells of the hair follicle bulge can be induced to form new hair follicles has been presented (Morris et al. 2004).

While neofolliculogenesis is not generally believed to occur normally in the adult state, new follicle formation can be induced experimentally by cellular manipulation. In early work Cohen (1961) showed that the isolated rat and guinea pig vibrissa papilla, a mesenchymal plug within the follicle base, could induce new follicle formation when experimentally implanted into the ear. In a series of now classical studies the laboratory of Oliver not only reproduced this work but also showed that the papilla could regenerate from the connective sheath surrounding the hair follicle (Oliver, 1966, 1967, 1970). Studying the same model Jahoda and his team cultured inductive papilla cells (Jahoda et al. 1984).

Studies of the cells which contribute to new follicle formation have been limited by the ability to assay these same cells for their hair follicle inductive, or trichogenic, properties. Attempts to develop trichogenic cell assays have been made in various experimental systems such as hanging drop cultures (Hardy 1949), granulation tissue beds (Reynolds & Jahoda 1992), collagenous shells (Reynolds & Jahoda 1994) and kidney capsule cultures (Takeda et al 1998, Inamatsu et al 1998). A valuable method for testing inductive cells was put forth by Lichti and her associates (Weinberg et al. 1993, Lichti et al., 1993) using an immunoincompetent mouse and silicon chambers. While the Lichti et al. assay is a dependable means for identifying trichogenic cells, it is demanding in terms of cell number, time and number of animals required.

In order to elucidate the mechanism of new hair follicle formation from dissociated cells, we set out to develop a more rapid mini-assay which would also faithfully reflect trichogenic properties. Described herein is an method or assay which uses many fewer cells (one million instead of 10 million) than the Lichti/Prouty assay, gives dependable results in less time (10 days instead of 35 days), and reduces the need for large numbers of mice (e.g. six or more assays can be performed in one mouse at one time). In the method of this invention we have found that placing trichogenic cells into the skin will within 8-12 days produce an array of follicles appearing as a cutaneous patch. Exposing the assay of hair follicles either by piercing, cutting or scratching the adjacent skin cover or by placing the cell in the superficial most dermis, produced egressing hair shafts.

### BRIEF DESCRIPTION OF THE DRAWINGS

Figure Legends

FIG. 1. Hair folliculoneogenesis after intracutaneous injection of dissociated epidermal and dermal cells.

(A) Phase contrast microscope picture of mouse neonatal dissociated dermal cells and epithelial buds or aggregates before injection into the recipient skin. Arrow points to an epithelial aggregate. (B) Patch skin as seen from the ventral side of the dissected out skin. The inset at the right shows posterior dorsal skin of the nude mouse depicting the circular black elevated patch (arrow) visible to the naked eye after two weeks. The inset on the left shows a low magnification view of the entire patch from the ventral side after dissection. Scale bars are 1 mm; (C, D) Histological view the hair patch region. (C) is a horizontal section showing an “infundibular cyst” containing shafts typical of dorsal mouse skin. (D) is a vertical section showing the location of the patch hairs in the host skin. PC: Panniculus camosus; Epi: host skin epidermis

FIG. 2. Intracutaneous injection of dissociated epidermal cells alone or dermal cells alone show no folliculoneogenesis.

The injected cells consisted of either epidermal buds alone (A and B) or dissociated dermal cells alone (C and D). Photomicrographs of the recipient nude mouse skin as seen from the ventral side of the skins (A and C) or by H & E histology (B and D).

FIG. 3. Evidence of hair follicle cycling in the patch assay.

Photomicrographs of patch assay in recipient nude mouse skin, as seen from the ventral side of the skin at A) day 13 where follicles are in the growing phase (anagen) or C) day 21 where follicles are in the resting phase (telogen). The respective histology is seen below in (B) and (D).

FIG. 4. Independence of patch hair follicle formation/cycle and recipient host skin follicle cycle.

Nude mouse skin in the hair patch region showing follicles in anagen within the patch region whereas the nude mouse host region shows follicles in the telogen phase (H & E histology).

FIG. 5. Effect of cell number and epithelial/dermal ratio on resultant hair formation

(A) Effect of total cell number injected on the number of follicles produced. A bar diagram showing the number of follicles generated in the patch assay in relation to the number of dermal cells (epidermal and dermal cell ratio was 1:100 in all cases) injected intracutaneously in nude mouse. (B) Effect of epidermal and dermal ratio on the number of follicles produced. A bar diagram showing the number of follicles generated in the patch assay in relation to varying ratios of the number of dermal cells to epidermal aggregates. The dermal cell number was fixed at 1 million for each injection. Bars represent s.d. from 4 samples.

FIG. 6. Surface extrusion of hair shafts.

(A) Effect of patch hair follicles transplanted into other sites. Picture was taken at two weeks after transplant (see Text for methods). (B) Effect of wound creation on the surface skin overlying a patch. Picture was taken 2 weeks after the wound was created. (C) Effect of inserting a tube into a patch. Picture was taken at 21 day after the initial injection.

FIG. 7. Morphogenesis and immunohistochemistry of hair follicles from day 1 through day 8 of the patch assay. Day 1:

(A) Histology of injection site showing small solid and discrete epithelial cell clusters (arrow) in a stroma of plump blastema-like cells. (B) Patch assay using GFP labeled epithelial cells and wild type dermal cells showing epithelial lineage of cell clusters (arrow). (C). TUNEL staining showing reactivity (arrowhead) amongst the single cells sparing the clusters. Day 2: (D) Patch assay showing vimentin stain of the stroma (E) Developed patch at a level just below the panniculus carnosus showing prominent epithelial clusters. (F) The clusters sit in a rich mucinous stroma (Colloidal iron stain). (G) High power of the epithelial clusters showing focal apoptotic cell necrosis (arrowhead). Microscopy of a cluster showing (H) central keratinization with some eccentricity of cell growth; (I) eccentric expression of EDAR(arrow), (J) eccentric placement of dividing cells(arrow), (K) peripherally expression of P63, (L) central keratinization, (M) eccentric expression of CD44(arrow), and (N) eccentric placement of progenitor papilla cells expressing alkaline phosphatase(arrow). Day 4: (O) Eccentric placement of dividing cells in the clusters(arrow); (P) Early follicle bud-like structures showing P63 expressing outer cells; (Q) MSX-2 expression in the periphery of the clusters (arrowhead) and in the surrounding stroma; (R) First expression of GATA3 a marker of the internal root sheath(arrow). (S) Oct4 staining in the area of sebaceous gland growth (arrow); (T) Eccentric versican staining in the early papilla (arrow). Day 6: (U) Infundibular cyst enlargement with projecting follicular forms (arrow). (V) In the GFP labeled epithelial cell lineage experiment the follicular papilla (arrow) is well defined at this time. (W) Papilla definition is also seen by alkaline phosphatase expression (arrow). Day 8: (X) Fully formed mature follicle present at this time. Insets show Oct 4 staining the sebaceous gland cells and CD 34 staining the cell in the sebaceous gland basal layer.

FIG. 8: Proposed Mechanism of Folliculoneogenesis from Dissociated Trichogenic Cells

In this figure is sketched the apparent steps of new follicle formation starting from dissociated cells (A). Very early after injection there is homotypic clustering of epithelial cells (B) followed by prominent apoptotic cell dropout (gray colored cells) in the clusters (C) with the formation of a “infundibular cyst” (D). Growing from the “infundibular cyst” at various poles the epithelial cells form follicular buds, pegs (E) and finally the mature follicle (F). Ultimately, the cycling follicles are destroyed leaving a foreign body reaction and scar.

The term ‘Hair Patch’ assay is used here to describe the morphological and molecular patterns of new follicle formation. This assay provides an easy and rapid determination of the effect, if any, of growth factors, cellular types, scaffolds, scaffold materials, pharmaceuticals or other internal or external influences have on upon new follicle formation. This work also underscores the role of an epithelial platform in new organ formation illustrated here by folliculoneogenesis.

### DETAILED DESCRIPTION OF THE INVENTION

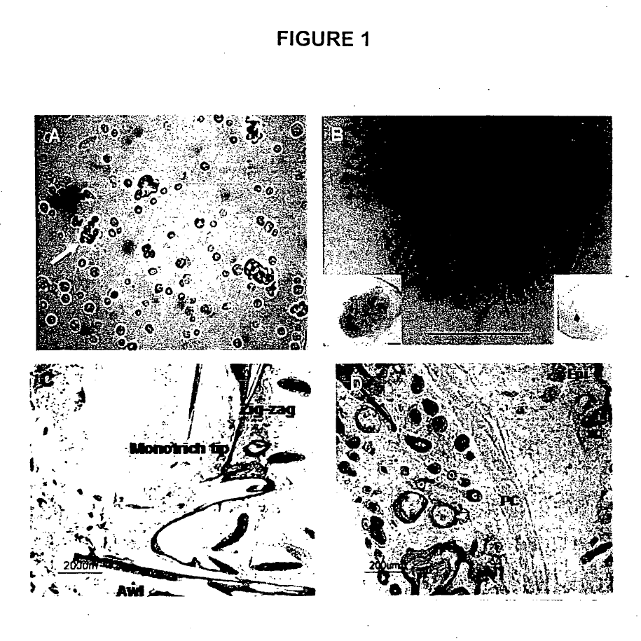

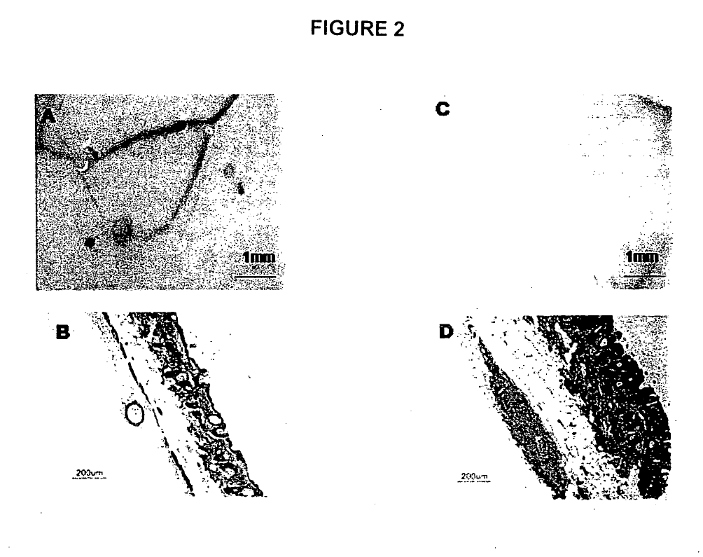

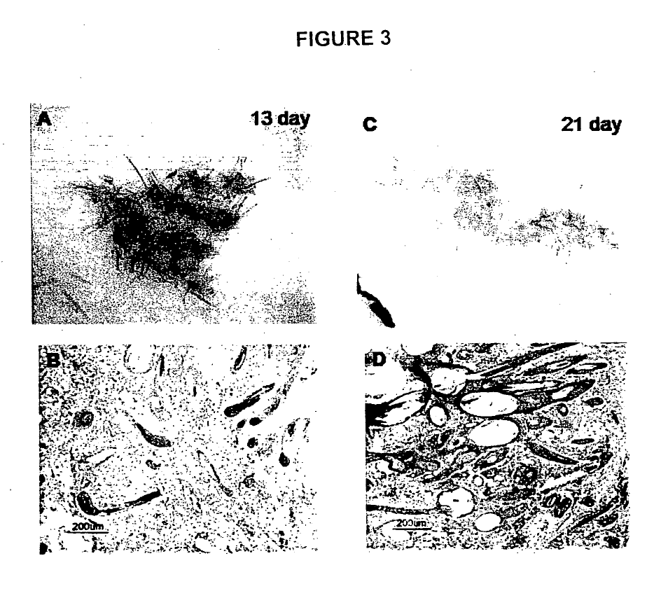

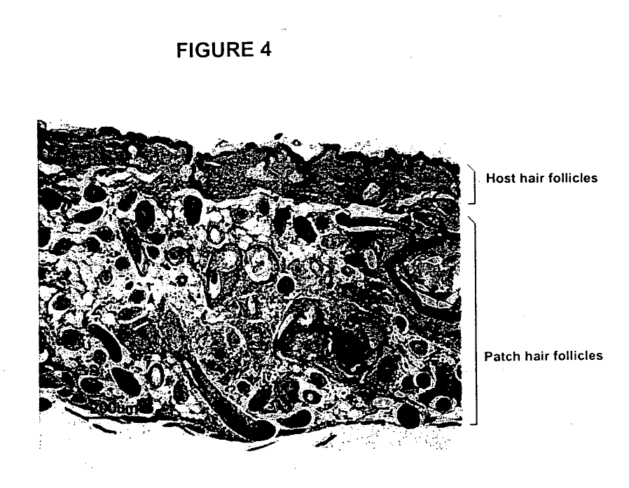

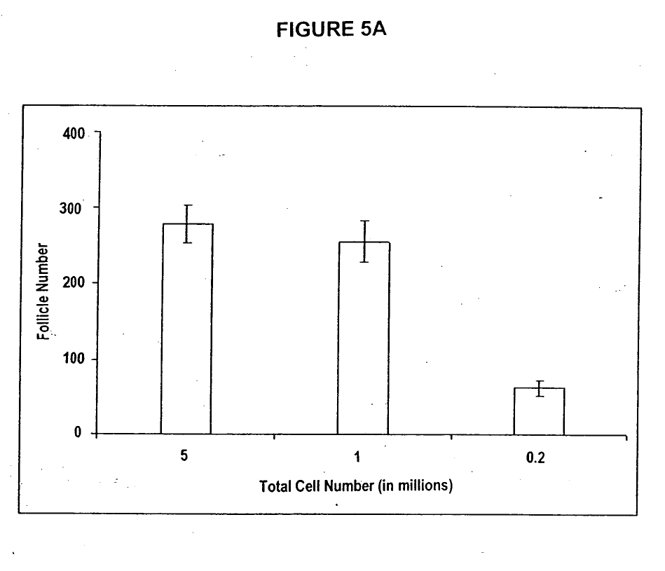

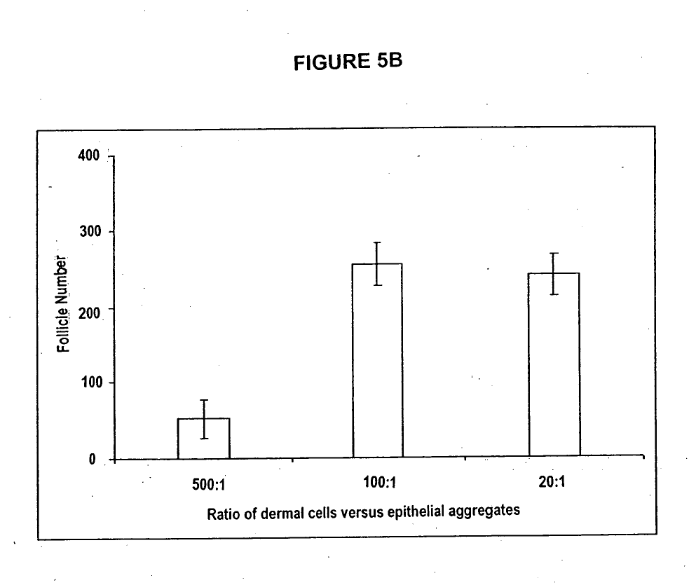

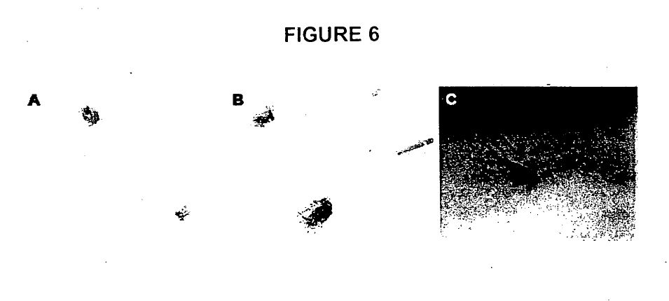

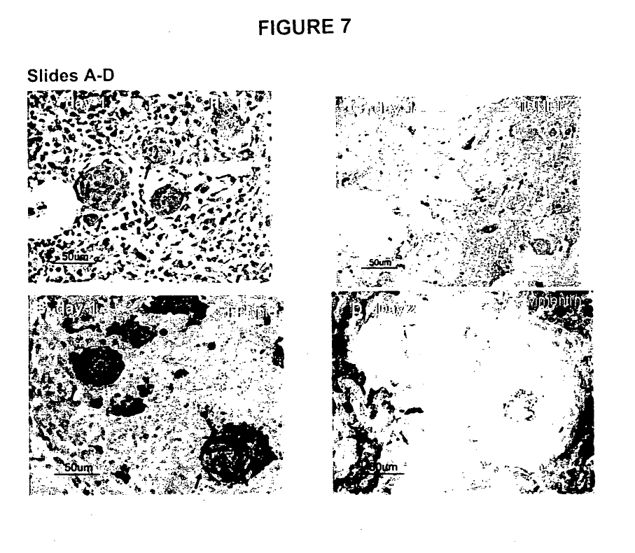

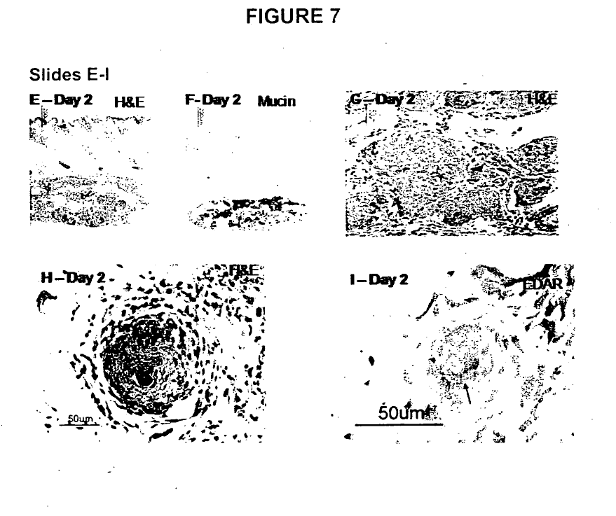

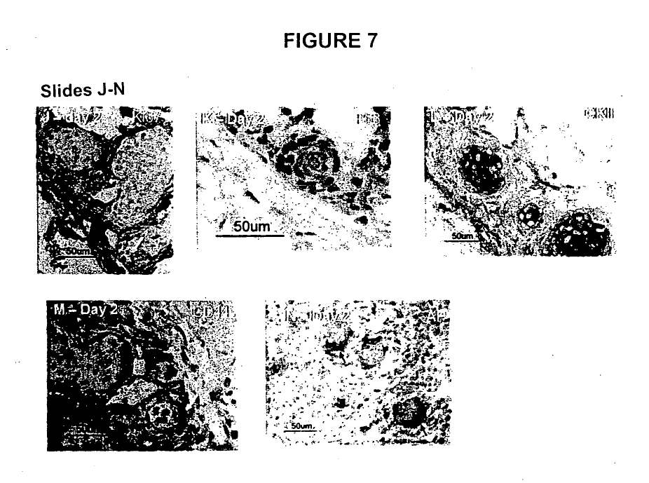

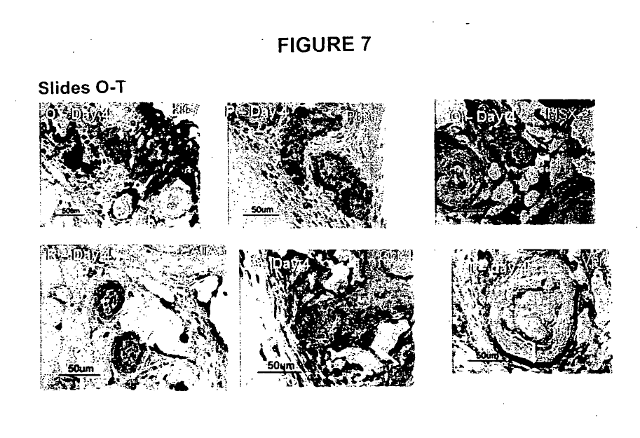

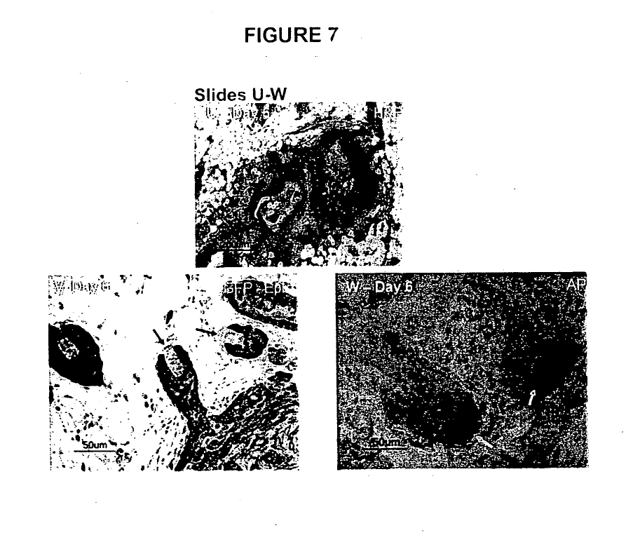

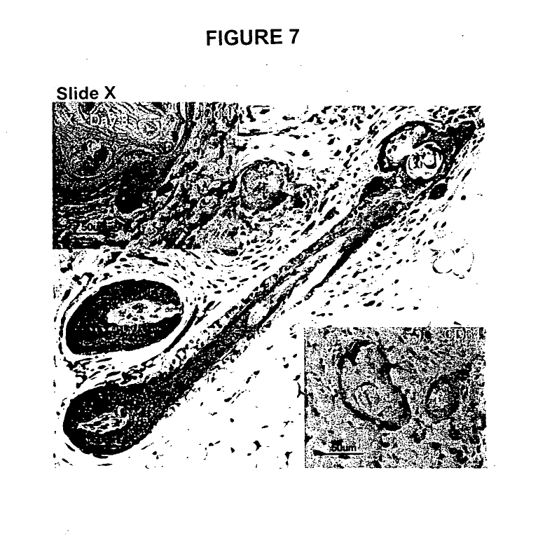

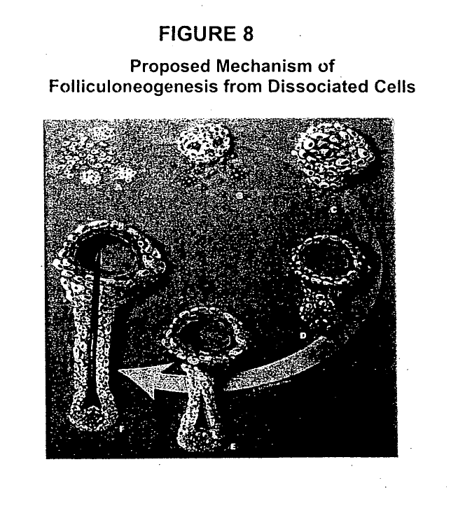

Hypodermal injection of trichogenic mouse cells into mouse skin leads to the rapid formation of hair follicles. When the same population of epithelial and mesenchymal cells, as used in the Lichti/Prouty assay (Weinberg et al., 1993; Lichti et al., 1993; Prouty et al., 1996), was injected directly into the skin, (instead of into a chamber), the rapid formation of mature hair follicles within the dermis was observed. The initial cell population used for implantation is composed of dissociated dermal cells and small clusters of epidermal cells derived from 0-2 day old neonatal mice (FIG. 1A). Routinely, the skin sites that had received the injected neonatal trichogenic cells, and referred to here as “the patch,” were harvested 12 days later. At this point the patch appears as a slightly elevated, gray, round area of skin (FIG. 1B right inset). Individual hair follicles are best visualized on the visceral side using a dissecting microscope (FIG. 1B and left insert). In a typical assay a cluster of about 200 hair follicles with associated shafts form at each site after injection of 1 million dermal cells and 10,000 epithelial aggregates.

As identified by cross section of hair shafts both tylotrich and underhair (awl, auchene, zig-zag) follicles form as one would expect since unfractionated pelage skin dermal cells are used in this preparation (Dry 1926) (FIG. 1B, C, D). This finding is consistent with previous studies indicating that the follicle type formed reflects the origin of the dermal component (Jahoda 1992).

There is some abnormal variation in the morphology of the follicles formed. Although many forms are identical to in situ follicles there are also follicle forms which show some distortion and irregular placement consisting of cystic dilation of the distal-most pilary canal, retention of hair shaft, and abnormally long telogen forms. Most hair follicles in the patch, however, lie parallel to the skin surface with the bulb (follicle base) centrifugally positioned. To determine the effect of dermal placement of trichogenic cells on new follicle formation we injected the same number of cells either into the hypodermis, or along the deeper-lying, facial plane, subcutaneously. Histologically, in the former case, cells were present in the hypodermis at the approximate level of the panniculus carnosus (below in FIG. 1D and above and within in FIG. 4). In this case the cells were confined to a small volume within the dermis and good follicle formation results. When the preparation was injected subcutaneously, upon the fascial plane, the cells spread over a larger area and few to no follicles formed. To examine if new hair formation is unique to immuno-incompetent mice such as the nude (nu/nu) mutant, we performed the patch assay in wild type adult C57BL/6 mice using newborn homogeneic cells. The homogeneic cells were tolerated by the adult mouse and patch hairs were seen at day 14 of injection. Thus, new hair formation in this system—in terms of morphology and time of development—is not unique to the immunoincompetent host.

Successful formation of follicles requires both epidermal and dermal cells. Injection of epidermal cells only leads to the formation of epithelial cysts with pigmentation (FIG. 2 A, B). Injection of dermal cells only produces a white patch of stroma at the injection site (FIG. 2C, D). The ratio of dermal cells to epidermal cells for successful follicle formation in this assay falls in the range of about 500:1 to about 1:100, preferably about 100:1 to about 1:20 and most preferably about 20:1 to about 1:2.

It is of interest that in most cases the mature patch assay rested on prominent host vessels as if the growth of this highly interacting metabolic system was angiogenic. Evidence for the angiogenic properties of the follicle has been presented (reviewed in Stenn & Paus 2001)

Patch Assay Follicles Cycle

Although we could see from the histological studies that the newly formed hair follicles enter anagen, we next asked the question if these follicles cycle beyond the first anagen. By harvesting the site at various times after injection we found that the population of newly formed follicles does indeed cycle in aggregate. In mice from which the trichogenic cells were derived, the normal growth, or anagen, phase of the cycle in vivo extends for about 18 days followed by the resting phase, telogen (Stenn & Paus 2001). As seen in FIG. 3A, the follicles in the patch are predominately growing (anagen) at 13 days, whereas they are resting (telogen) at 21 days (Many elongated telogen forms are found but no anagen bulbs are present at this time). This observed cycle correlates well with the time course of the first neonatal hair cycle (Paus et al 1999). At 40 days anagen follicles are again found; however, when the patch tissues were harvested at later time points (3-4

month), we found pigment deposits, epidermoid cysts, foreign body reactions and focal fibrosis. We interpreted these changes as secondary to the fact that as the formed shaft was not properly shed; it remains in the dermis to incite an inflammatory and foreign body reaction.

Since the whole mouse skin organ undergoes dramatic changes over the cycle (Chase et al 1953, Hansen et al 1984), we asked whether the hair cycle of the recipient skin corresponds to that in the patch follicles. When examining patch follicles, we found patch hairs in anagen while the host skin hair follicles were in telogen (FIG. 4). These observations collectively suggested that the internal clock of follicle cycling is inherent in the constituent trichogenic cells and not dependent on the host skin hair cycle.

Since early studies suggested that the number of follicles forming in a given assay is a function of the total number of cells delivered and the ratio of epidermal to dermal cells, we sought to optimize this relationship. To do this we assayed various numbers of dermal or epidermal cells. Increasing the dermal cells five fold to 5 million did not produce more hair follicles (278±25) compared to the injection of 1 million dermal cells (255±28, FIG. 5A). On the other hand, when the number of dermal cells was reduced 5 fold to 0.2 million, the number of hair follicles formed, compared to the case with 1 million dermal cells, was significantly reduced (63±10). These data are interesting since the ratio of epidermal aggregates to dermal cells in all three situations was maintained at 1:100. When the dermal cells were fixed at 1 million and the number of epidermal aggregates were varied from 10,000 to 50,000, a comparable number of hair follicles formed over this range without any significant difference (255±28 and 240±27, respectively); however, the number of follicles formed was reduced by more than half (52±25) when epidermal aggregates were decreased to 2,000 (FIG. 5B). In all subsequent studies each patch assay was initiated using one million dermal cells in a dermal to epidermal ratio of 100:1.

Patch Hair Can Grow Out of the Skin Surface

To demonstrate that patch hair shafts formed underneath the skin surface could also grow out of the skin, we used several approaches (see Methods for details). For the first we harvested patch hair from one mouse at day 14, divided the patch into small pieces each containing a number of follicles at different orientation, and transplanted them into another nude mouse. The results showed that hair follicles with the right orientation when planted (bulbs inside the skin) could survive and grow out of the surface of skin (FIG. 6A). We have also created a channel to the skin surface to liberate patch hair shafts using two methods. One was to cut a shallow wound on top of a patch to expose the hair follicles (FIG. 6B) and the other was to insert tubing into a patch injection site and remove the tubing after 3 days (FIG. 6C). All of these methods liberated a tuft of hair growing out of the skin surface. We have examined the histology of the outgrown hair and found that they were at telogen at day 21 after initial injection, and re-entered anagen at about 4 weeks as manifest by anagen follicles at that stage. These results indicated that the patch hairs can grow out of the skin surface if an opening is created, and that they are able to go through a normal hair cycle.

New Follicle Formation From Dissociated Cells Involves Steps of Initiation, Morphogenesis and Differentiation and Starts from an Epithelial Platform.

The above studies indicate that the patch assay reproducibly generates mature cycling follicles. The next question of this hair follicle organogenesis system is how a new organ generates starting with dissociated cells. To perform these studies we assessed the patch assay over time by histological and immunochemistry. This study was repeated three times, once with wild type cells and twice with GFP labeled cells; the data were similar.

At one day after injection of the combined dermal and epithelial cells (see FIG. 1A), tissue sections show epithelial cell aggregates surrounded by plump mesenchymal cells reminiscent of blastema cells found in the regenerating amphibian limb bud (FIG. 7A; Tsonis 1996). The cell clusters are predominantly epithelial (FIG. 7A, B) as inferred from reciprocal experimentation involving epidermal (FIG. 7B) or dermal cells (not shown) from GFP mice in combination with C57B1/6 mouse cells and by vimentin stain (FIG. 7D). This is confirmed by pan-cytokeratin-II antibody stain (FIG. 7L). Although the cell clusters showed little mitotic activity at this time (as shown by Ki67 data, not shown), they continued to grow apparently by aggregation (compare FIG. 7B with FIG. 7G). An interesting feature of early morphogenesis, as seen as early as day one after cell placement, is the prominent apoptosis observed amongst the delivered cells (FIG. 7C). This apoptotic activity appears extensively within the stroma but is also found within the epithelial clusters. It is most intense at day 1 decreasing thereafter.

By two days the cells are embedded in a glycosaminoglycan-rich stroma (FIG. 7F). The epithelial clusters show focal asymmetric growth. At this time there is eccentric placement of 1) dividing cells as evidenced by Ki67 stain (FIG. 7J), 2) of EDAR (Pispa & Thesleff 2003) immunoreactivity (IR) in some clusters suggestive of placode formation (FIG. 7I), and 3) early mesenchymal condensation as observed by H & E staining, CD44 IR, and alkaline phosphatase (Handjiski et al 1994) (FIG. 7H, 7M, & 7N). Some of the epithelial clusters at this time now show focal prominent apoptosis with central keratinization (FIG. 7G). Others show central cyst formation where the cells lining the cyst contain keratohyalin granules (FIG. 7H). The resultant structure is highly reminiscent of the infundibular (most distal) portion of the hair follicle. These “infundibular cysts” serve as the platform from which the incipient follicles grow. In these clusters the expression of p63 (FIG. 7K), a p53 analog and marker of the adnexal placode, and a required structure for epidermal adnexal development, (Mills et al 1999, Yang et al 1999) and pan-cytokeratin-II (FIG. 7L), a keratinization differentiation marker (Coulombe et al 2002), appear to be mutually exclusive, with p-63 IR more towards the periphery.

At 4 days some of the cluster fusions are prominent with a central cystic space. Extending from these structures are follicular germs, buds and early peg stage, as seen by H & E stain and epithelial cell specific IR of GFP(data not shown), and Ki67, and p63 IR( FIG. 7O, FIG. 7P). Msx-2, known to be expressed in hair follicle placode ectoderm and subsequently in epithelial matrix cells (Reginelli et al 1995; Ma et al 2003) and versican, a papilla marker (du Cros et al 1995) are expressed eccentrically towards the budding follicle (FIG. 7Q, FIG. 7T), where GATA3-IR, an inner root sheath marker (Kaufman et al. 2003) is observed first at this time (FIG. 7R). In addition, Oct4 IR, a marker of pluripotent embryonic stem cells ( Nichols et al 1998) was limited to a few cells in the matrix of budding follicles in the vicinity of the forming sebaceous gland (FIG. 7S).

More mature follicles with distinct papilla as seen by H & E (FIG. 7U) and with epithelial specific GFP IR (FIG. 7V) or papilla specific expression of alkaline phosphatase activity are observed by day 6 (FIG. 7W). Early follicular melanocytic pigmentation is seen at this time

At 8 days full mature follicles are present growing from the infundibular cystic structures (FIG. 7X). Sebaceous glands are developed by this time and displayed Oct4 IR. CD34 IR was expressed in cells, surrounding sebaceous gland, which could originate from cells of bulge origin (Trempus et al 2003). It is notable that during the first week of morphogenesis, follicles are not all of one morphogenetic form: a range of forms are present. For example, according to the classification system of Paus et al (1999) follicles in phases IV, V and VI were present at day 8. In some follicle structures sebaceous gland formation but no shaft formation was seen.

The system described here shows the rapid formation of new hair follicles organs on combining isolated epithelial and mesenchymal cells. The pattern of organogenesis presented suggests a morphological sequence as sketched in FIG. 8. In the earliest phase there is epithelial cell aggregation and fusion of individual aggregates, within a very rich mucinous cellular dermal stroma. This is followed by a phase of apoptotic remodeling of the clusters (the shaping apoptosis or Mode 1A of Chang et al 2004) to form an epithelial structure very similar to the infundibulum of the follicle, an “infundibular cyst”. Over the course of follicle growth these cystic structures fuse to form larger and larger cysts. Evidence that the earliest cysts are asymmetric comes from the eccentric placement of the dividing cells (Ki67), the position of the early papilla (versican, alkaline phosphatase, CD44), and the location of placode markers (EDAR, P63). By 4 days there is hair follicle germ cell growth from the periphery of the “infundibular cyst” to form early bud and then peg structures (Paus et al. 1999). Completely mature, fully differentiated new hair follicles and shafts can be seen histologically within 6 to 8 days and by naked eye within 14 days.

One of the fundamental observations of this study is the role of an epithelial platform in the earliest phase of new follicle formation. In many other developmental epithelial-mesenchymal interacting systems the first morphogenetic event is associated with an epithelial platform. This epithelial platform has many homologues in biology: the apical ectoderm ridge in the forming limb bud (Gilbert 2000), the wound epithelium in the regenerating limb (Tsonis 1996), epithelial condensate of the tooth bud (Arias & Stewart 2002) and the placode of the feather and hair follicle (Widelitz and Chuong 1999). In forming a new follicle from dissociated cells the epithelial cells quickly cluster and then remodel themselves to generate a structure highly reminiscent of the primitive epidermis with its placode, and the acral hair follicle with its infundibulum; it is both of these structures which support new follicle formation in the mouse newborn and adult, respectively. After the formation of this “infundibular cyst”, polar placement of mesenchymal condensates and cycling epithelial cells lead to the early recognizable hair follicle germ and from these sites the hair follicle anlagen, the bud and peg forms, result. The completely mature follicle has all the elements of the mature in situ follicle including a normal appearing sebaceous gland; moreover, it undergoes a cycle with its unique stages and periodicity. The limitation of this system is that follicle growth in this environment is finite: because there is no means of disposing the shaft and its keratinous product, the environment fills with inflammatory, foreign body and fibroblastic cellular elements with eventual follicle ablation.

As discussed above, while other systems for generating hair follicles from dissociated cells have been described, none is as efficient in terms of time, cells, and animal usage. We have been surprised how rapidly new hair follicle formation occurs in this ‘Hair Patch’ system. As seen in the morphogenetic studies (FIG. 7), mature follicles with shaft formation occur within 8 days. This rate of formation corresponds very closely to the in vivo situation of the newborn mouse (Paus et al 1999). It is interesting that new shaft formation from a transplanted follicle requires about 45-70 days (Hashimoto et al., 1996), so that, while counterintuitive, starting from an organized structure takes a longer period of time to regenerate than forming a new follicle from dissociated cells. In the latter case, though, the transplanted intact anagen follicle apparently must undergo a regression process and then reform its cycling portion (Hashimoto et al 1996).

Although we conducted studies to optimize the ratio of epithelial to dermal components and the number of cells to deliver, we have not yet been able to establish a minimal cell number for generating a single follicle. We have routinely found that placement of one million trichogenic cells (100:1 ratio of dermal to epidermal cells) into the dermis will result in about 200 hair follicles. This translates into the estimate of 5000 cells per newly formed follicle. In view of the fact that there is extensive apoptotic remodeling in the early phases of folliculoneogenesis, and given the fact that all cells may not be endowed with trichogenic capability, it is conceivable that many fewer cells are actually contributing to a given follicle.

We found that the success of this assay is dependent on the placement of the trichogenic cells into a small space. The dermis/hypodermis appeared to work because the tissues are normally not loose and provide a compact environment for the interacting epithelial and dermal cells. If, on the other hand, the cells were placed on the subcutaneous fascial plane, few if any follicles resulted. We interpret this finding to imply that the formative trichogenic cells must be kept in close contact; we have not excluded, though, the possibility that the dermis itself offers a unique milieu. The advantage of this restricted space requirement is that as many as 8 assays could be performed on each mouse, reducing animal usage and cage requirements.

It was surprising and unexpected that dissociated trichogenic cells rapidly reform follicles but that the newly formed follicles cycle; moreover that the newly formed follicle cycle has a period very close, if not exactly matching, the cycle of the derivative follicles. The phenomenon of hair cycling appears to be inherent in the structure of the follicle; in other words, if a follicle forms, it will cycle—the cycling trait seems to be inherent in the follicle structure itself. In an incidental observation it was of interest to notice that the formation and cycling of the reformed follicles occurred independently of the host follicle cycle. Since early studies indicated that the whole skin organ is affected and changes with each phase of the cycle (Chase et al 1953, Hansen et al. 1984), it was not clear if telogen skin could support anagen follicle growth. If telogen epidermis actually does produce an inhibitor to anagen growth, then that inhibitor may not diffuse long enough distances (Paus, et al. 1990) to reach the cells in the patch reaction (Gurdon 1989).

The morphology of the follicles formed is in general similar to intact follicles; however, there may be variation in follicle forms seen at any one time. This variation may be apparent in the cycle phase, the size of the infundibular cyst platform from which the follicle grows and the variation in the size of the follicle itself. As demonstrated by the low power microscopic pictures of the patch assay (see FIG. 1B) the orientation of the newly formed follicles is, in general, with the bulb, or proximal end of the follicle, located toward the periphery. While this finding was not analyzed in detail the observation suggests that the follicle base might have unique requirements—such as blood supply—forcing the highly metabolic and dividing end toward a more favorable environment. As described above the patch assay rests on prominent host vessels. It is notable that other epithelial-mesenchymal interacting systems demand new vessel formation in order to progress (Schwarz et al 2004) and it is probably true in this situation as well recognizing the angiogenic associations of the hair follicle (Stenn and Paus 2001). At the end of the first cycle although the population of follicles in these preparations reaches telogen, all the follicles do not attain typical telogen morphology. A telogen form with a very long inferior portion is present (see FIG. 3D). We do not completely understand the meaning of this abnormal telogen form but as the latter abnormal form is very similar to the telogen forms seen in the asebia mouse (Sundberg et al. 2000) it may be that such forms occur when there is difficulty in expelling the shaft.

An interesting observation we noticed was IR of Oct4 in matrix cells and sebaceous glands of budding follicles at days 4-8. Although recently Oct4 expression has been observed in presumptive stem cells derived from porcine skin (Dyce et al., 2004), to the best of our knowledge this is the first report of Oct 4 expression in hair follicle, specifically the sebaceous gland and its anlage. Oct4 is important for embryogenesis but was known to be expressed only in germ cells of adult animals (Nichols et al., 1998; Scholer et al., 1989).

In summary, we describe a system here that can serve as an assay for trichogenic cells and as a model for studying the morphologic and molecular mechanisms of new organ formation from dissociated cells. Using this system we found that dissociated cells very early in the process construct an epithelial platform which is shaped by apoptosis in order to set the stage for hair germ formation; eventually a mature cycling pilosebaceous structure results.

### EXAMPLES

Preparation of Neonatal Mouse Hair Follicle Progenitor Cells

Mice were purchased from either Charles River, Wilmington, Mass. (pregnant C57B1/6 mice) or from Jackson Laboratories, Bar Harbor, Me. {Green Florescent Protein (GFP) mice [FVB.Cg-Tg(GFPU)5Nagy/J]}. Cell preparations followed an adaptation of the procedure of Prouty et al (1996). Briefly, mice were housed in the University of the Sciences in Philadelphia (USP) animal facility, 12 hour light and dark cycles, fed with animal chow (Purina Rodent Lab Diet #5001) and water ad libitum. Following USP IACUC approved protocol, truncal skin was removed from newborn mice and rinsed in Ca⁺⁺ and Mg⁺⁺ free PBS. The skin was laid flat in PBS containing Dispase (2.5 mg/ml, Invitrogen, Carlsbad, Calif.) at 4° C. overnight or at 37° C. for 2 hrs. Subsequently, inductive dermal cells and epidermal aggregates were isolated as previously described (Weinberg et al., 1993, Lichti et al 1993, Prouty et al., 1996). Cells were used either the same day or kept frozen at −80° C. for future use (epidermal cells frozen in Synth-a-Freeze® Cryopreservation Medium, Cascade Biologics, and dermal cells frozen in medium A, Prouty et al 1996, containing 5% DMSO and 10% bovine serum).

Recipient Mice and Cell Delivery for Follicle Morphogenesis in the Patch Assay

Trichogenic cells were assayed in male nude (nu/nu) mice (Charles River, Wilmington, Mass.) at 7-9 weeks of age. Following USP IACUC approved protocol, mice were anesthetized (ketamine, 100 mg/kg, Fort Dodge Animal Health, Iowa/xylazine, 10 mg/kg, Phoenix Scientific Inc., St. Joseph, Mo.). Unless otherwise stated for each intracutaneous injection, 1×10⁶ dermal cells and 10,000 epidermal aggregates were resuspended (50-70 μl of DMEM-F12 medium; Invitrogen, Carlsbad, Calif.) and injected (25 gauge needle) into the hypodermis of the mouse skin, forming a bleb. The injection site was marked by a black tattoo puncture (242 Permanent Black Pigment, Aims, Hornell, N.Y.). The number of hair follicles formed in a given patch assay was quantified by microscopic photography and morphometry; hair follicle count was executed by three separate observers.

Outgrowth of Patch Hair.

We used three approaches to test if hair shafts produced within the patch assay could grow out of the skin surface and were morphologically normal. 1) In the first, regenerated follicles and the surrounding tissue from a 12 day or later patch assay was dissected out and the patch was cut into small fragments, each containing a cluster of hair follicles. An 18 G needle was used to create several channels in the skin of a different nude mouse, and patch assay fragments suspended in PBS were inserted into the channels. 2) In the second method, using a pair of scissors a shallow wound was made in the skin overlying a mature patch assay (day 12); the wound was then covered with adhesive bandage for two days after which the bandage was removed. 3) In the third method, a segment of polyurethane intravascular tubing (Instech Solomon, Part No: BPU-T20, 2-3 French in diameter) was threaded into and out of the skin overlying and into a patch assay site (tube insertion on day 2 after injection; tube removal on day 4-5 after injection). The presence of shaft outgrowth was recorded daily.

Histology and Immunohistochemistry

Mouse skins were harvested and fixed in 10% formalin overnight. After paraffin embedding the tissues were processed for H&E histology (Presnell et al 1997). For immunohistochemistry, dewaxed sections were processed for antigen retrieval by heating in 10 mM sodium citrate (pH 6.0) at 98° C. for 10-15 min prior to incubation with primary antibody. The following primary antibodies were used at the indicated dilutions or concentrations: GFP (Novus Biologicals, Littleton, Colo., 1:200); Ki67 (BD Biosciences Pharmingen, San Diego, Calif., 1:10); p63 (BD Biosciences Pharmingen, San Diego, Calif., 4 μg/ml ); CD44 (Chemicon, Temecula, Calif., 15 μg/ml); CD34 ( MEC14.7; Novus Biologicals, Littleton, Colo., 1:10); Pan-Cytokeratin-type 11, CK-11 (Chemicon, Temecula, Calif., 1:200); Versican (Chemicon, Temecula, Calif.,10 μg/ml ); Msx2 (Santa Cruz Biotechnology, Inc, Santa Cruz, Calif., 1:50): Oct4 (Chemicon, Temecula, Calif., 20 ug/ml); GATA3 (Santa Cruz Biotechnology, Inc, Santa Cruz, Calif., 1:50). Vimentin (Chemicon Intl. 5 μg/ml). Formalin fixed paraffin embedded sections were used for all immunohistochemistry except that for anti-EDAR where frozen sections were used after acetone fixation of 2 minutes at −20° C. Primary antibodies were detected by biotinylated secondary antibodies followed by incubation with streptavidin-peroxidase complex and aminoethyl carbazole (AEC) chromogen (Histostain-SP Kit, Zymed Laboratories, San Francisco, Calif.).

Alkaline Phosphatase and Apoptosis Staining

Tissues in Tissue-Tek O.C.T. Compound (Electron Microscopy Sciences, Ft. Washington, Pa.) were frozen in dry ice and 4 μM cryosections were fixed in 4% paraformaldehyde/PBS for 20 min, washed in PBS and incubated for 15 min in the developing solution routinely used for alkaline phosphatase (Histostain SAP Kit, Zymed Laboratories, San Francisco, Calif.). Formalin fixed and Paraffin embedded sections were processed for TUNEL staining using DermaTACS In-situ Apoptosis Detection Kit (Trevigen, Gaithersburg, Md.) following manufacturer's instructions.

### REFERENCES

ARIAS, A M and STEWART A. (2002) Molecular Principles of Animal Development Oxford University Press, Oxford.
ATALA A, & LANZA R P, eds (2002). Methods of Tissue Engineering, Academic Press, New York.
ATALA A. (2004) Tissue engineering and regenerative medicine: concepts for clinical application. Rejuvenation Res 7:15-31.
CHANG C-H, Yu, M, Jiang T-X, Yu H-S, Widelitz B R, & Chuong C-M. (2004) Sculpting skin appendages out of epidermal layers via temporally and spatially regulated apoptotic events. J Invest Dermatol 122:1348-1355.
CHASE H B, Montagna W, & Malone J D. Changes in the skin in relation to the hair growth cycle.(1953) Anat Rec 116:75-82.
COHEN J. (1961) The transplantation of individual rat and guinea-pig whisker papillae. J Embryol Exp Morphol 9:117-127.
COTSARELIS G, Sun TT, Lavker R M. (1990) Label-retaining cells reside in the bulge area of pilosebaceous unit. Implications for follicular stem cells, hair cycle and skin carcinogenesis Cell 61:1329-1337.
COULOMBE P A, Omary M B. (2002) ‘Hard’ and ‘soft’ principles defining the structure, function and regulation of keratin intermediate filaments. Curr Opin Cell Biol. 14:110-122.
DRY F W (1926) The coat of the mouse (Mus musculus). J Genetics 16:281-340, 1926.
DU CROS D L, LeBaron R G, Couchman J R. (1995) Association of versican with dermal matrices and its potential role in hair follicle development and cycling. J Invest Dermatol. 105:426-31.
DYCE P W, Zhu H, Craig J, Li J. (2004) Stem cells with multilineage potential derived from porcine skin. Biochem Biophys Res Commun. 316:651-658.
GHARZI A, Reynolds A J & Jahoda C A (2003) Plasticity of hair follicle dermal cells in wound healing and induction. Exp Dermatol 12:126-136.
GILBERT S F. (2000) Developmental Biology, Sinauer Associates, Inc., Sunderland, Mass. pg 504-521.
GURDON J (1989) The localization of an inductive response. Development 105:27-33.
HANDJISKI B K, Eichmuller S, Hofmann U, Czametzki B M, Paus R. (1994) Alkaline phosphatase activity and localization during the murine hair cycle.
BR J DERMATOL. 131:303-310.
HANSEN L S, Google J E, Wells J Charles M W. (1984) The influence of the hair cycle on the thickness of mouse skin. Anat Rec 210:569-573.
HARDY M. (1949) The development of mouse hair in vitro with some observations on pigmentation J Anat 83:364.
HASHIMOTO T, Kazama T, Ito M, Urano K, Katakai Y & Yeyama Y. (1996) Histological examination of human hair follicles grafted into severe combined immunodeficient (SCID) mice. In Hair Research for the Next MILLENIUM EDS, D J J Van Neste and V A Randall, Elsevier Science BV, Amsterdam pg 141-145.
INAMATSU M, Matsuzaki T, Iwanari H, & Yoshizato K. (1998) Establishment of rat dermal papilla cell lines that sustain the potency to induce hair follicle from afollicular skin J Invest Dermatol 111: 767-775.
JAHODA CAB, Home K A & Oliver R F. (1984) Induction of hair growth by implantation of cultured dermal papilla cells. Nature 311:560-562.
JAHODA CAB. (1992) Induction of follicle formation and hair growth by vibrissa dermal papillae implanted into rat ear wounds: vibrissa-type fibres are specified. Development 115:1103-1109.
JAHODA C A, & Reynolds A J. (2001) Hair follicle dermal sheath cells: unsung participants in wound healing Lancet 358:1445-1448.
JAHODA CAB, Whitehouse C J, Reynolds A J, & Hole N. (2003) Hair follicle dermal cells differentiate into adipogenic and osteogenic lineages. Exp Dermatol 12:849-859.
KAUFMAN C K, Zhou P, Pasolli H A, Rendl M, Bolotin D, Lim K C, Dai X, Alegre M L and Fuchs E. (2003) GATA-3: an unexpected regulator of cell lineage determination in skin. Gene Dev 17:2108-2122.
KISHIMOTO J Ritsuko E, Wu L, Jiang S, Jiang N, & Burgeson R. (1999) Selective activation of the versican promoter by epithelial-mesenchymal interactions during hair follicle development. Proc Natl Acad Sci USA 96:7336-7341.
LAKO M, Armstrong L, Cairns P M, Harris S, Hole N, & Jahoda C A. (2002) Hair follicle dermal cells repopulate the mouse haematopoietic system. J Cell Sci 115:3967-3974.
LICHTI U, Weinberg W C, Goodman L, Ledbetter S, Dooley T, Morgan D, & Yuspa SH. (1993) In vivo regulation of murine hair growth: insights from grafting defined cell populations onto nude mice. J invest Dermatol 101:124S-129S.
M A L, Liu J, Wu T, Plikus M, Jiang T X, Bi Q, Liu Y H, Muller-Rover S, Peters H, Sundberg J P, Maxson R, Maas R L, Chuong C M. (2003) ‘Cyclic alopecia’ in Msx2 mutants: defects in hair cycling and hair shaft differentiation. Development 130, 379-389.
MILLS, A. A., Zheng, B., Wang, X.-J., Vogel, H., Roop, D. R., and Bradley, A. (1999) p63 is a p53 homologue required for limb and epidermal morphogenesis. Nature 398: 708.
MORRIS R J, Liu Y, Marles L, Yang Z, Trempus C, Li S, Lin J S, Sawicki J A, & Cotsarelis G. (2004) Capturing and profiling adult hair follicle stem cells. Nature Biotechnology 22:411-417.
NICHOLS J, Zevnik B, Anastassiadis K, et al. (1998) Formation of pluripotent stem cells in the mammalian embryo depends on the POU transcription factor Oct4. Cell. 95:379-391.
OLIVER R F. (1996) Whisker growth after removal of the dermal papilla and lengths of follicle in the hooded rat. J Embryol Exp Morph 15:331-347.
OLIVER R F. (1997) Ectopic regeneration of whiskers in the hooded rat form implanted lengths of vibrissa follicle wall. J Embryol Exp J Morph 17:27-34.
OLIVER R F. (1970) The induction of hair follicle formation in the adult hooded rat by vibrissa dermal papillae. J Embryol Exp Morphol 23:219-236.
OSHIMA H, Rochat A, Kedzia C, Kobayashi K & Barradon Y. (2001) Morphogenesis and renewal of hair follicles from adult multipotent stem cells. Cell 104:233-245.
PAUS R, Stenn K S, & Link R E. (1990) Telogen skin contains an inhibitor of hair growth. Brit J Dermatol 122:777-784.
PAUS R, Muller-Rover S, van der Veen C, Maurer M, Eichmuller S, Ling G, Hofmann U, Foitzik K, Mecklenburg L & Handjiski B. (1999) A comprehensive guide for the recognition and classification of distinct stages of hair follicle morphogenesis. J Invest Dermatol 113:523-532.
PISPA J and Thesleff I. (2003) Mechanisms of ectodermal organogenesis. Dev Biol. 262:195-205.
PRESNELL J K, Schriebman M, & Humason G L. (1997) Humason's Animal Tissue Techniques. Johns Hopkins Univ Press, 572pp
PROUTY S M, Lawrence L, Stenn K S. (1996) Fibroblast-dependent induction of a murine skin lesion with similarity to human common blue nevus. Amer J Path 148:1871.
PROUTY S M, Lawrence L, Stenn K S. (1997) Fibroblast-dependent induction of a skin hamartoma: Murine lesion with similarity to human nevus sebaceous of Jadassohn. Lab Invest 76:179-189.
REGINELLI,A D., Wang, Y-Q., Sassoon, D., and Muneoka, K. (1995) Digit tip regeneration correlates with regions of Msx1 (Hox 7) expression in fetal and newborn mice. Development 121: 1065-1076.
REYNOLDS A J & Jahoda CAB. (1994) Hair follicle reconstruction in vitro. J Dermatol Sci 7 (Suppl) S84-S97.
REYNOLDS A J & Jahoda C A B. (1992) Cultured dermal papilla cells induce follicle formation and hair growth by transdifferentiation of an adult epidermis. Development 115:587-593.
SCHOLER H R, Hatzopoulos A K, Balling R, Suzuki N, Gruss P. (1989) A family of octamer-specific proteins present during mouse embryogenesis: evidence for germline-specific expression of an Oct factor. EMBO J. 8:2543-2550.
SCHWARZ M A, Wan Z S, Liu J, & Lee M K. (2004) Epithelial-mesenchymal interactions are linked to neovascularization. Amer J Respir Cell Mol Biol 30:784-792.
STENN K S & Paus R. (2001) Controls of hair follicle cycling. Physiol Rev 81:449-494.
SUNDBERG J P, Boggess D, Sundberg B A, Eilertsen K, Parimoo S, Filippi M & Stenn K. (2000) Asebia-2J(Scd1(ab2J)): a new allele and a model for scarring alopecia. Amer J Path 156:2067-2075.
TAKEDA A, Matsuhashi S, Shioya N, & Ihara S. (1998) Histodifferentiation of hair follicles in grafting of cell aggregates obtained by rotation culture of embryonic rat skin. Scand J Plast Reconstr Hand Surg 32:359-364.
TAYLOR G, Lehrer M S, Jensen P J, Sun T T & Lavker R M (2000) Involvement of follicular stem cells in forming not only the follicle but also the epidermis. Cell 102:451-461.
TREMPUS C S, Morris R J, Bortner C D, Cotsarelis G, Faircloth R S, Reece J M, Tennant R W. (2003) Enrichment for living murine keratinocytes from the hair follicle bulge with the cell surface marker CD34. J Invest Dermatol. 120:501-11.
TSONIS P A (1996) Limb Regeneration, Cambridge Univ Press, Cambridge, 1996, 241 pp
WEINBERG W C, Goodman L V, George C, Morgan D L, Ledbetter S Yuspa S H & Lichti U. (1993) Reconstitution of hair follicle development in vivo: determination of follicle formation, hair growth, and hair quality by dermal cells. J Invest Dermatol 100:229-236.
WIDELITZ R B & Chuong C-M. (1999) Early events in skin appendage formation: Induction of epithelial placodes and condensation of dermal mesenchyme. J Invest Dermatol Sympos Proc 4:302-306.
YANG, A., Schweitzer, R., Sun, D., et al.(1999) p63 is essential for regenerative proliferation in limb, craniofacial and epithelial development. Nature 398: 714.

What is claimed is as follows:

1. A method of inducing hair follicle formation from dissociated cells comprising the steps of:
a) providing a mixture of dissociated cells comprising dermal cells and epidermal cells;
b) injecting the mixture into a dermis/hypodermis of a mammal producing a dermal bleb; and
c) permitting the injected cellular mixture to grow a new hair shaft.

2. A method according to claim 1 wherein the ratio of dermal cells to epidermal cells falls in the range of about 100:1 to about 1:20.

3. A method according to claim 1 wherein the ratio of dermal cells to epidermal cells falls in the range of about 20:1 to about 1:2.

4. A method according to claim 1 further including the step of permitting the newly formed hair shafts to egress by disrupting the dermis/hypodermis adjacent to the hair shaft.

5. A method according to claim 4 wherein the disruption occurs by cutting the dermis/hypodermis.

6. A method according to claim 4 wherein the disruption occurs by inserting a hollow tube through the dermis/epidermis removing the tube after a period of healing and permitting the hair follicles to egress through skin where the tube previously was located.

7. A method according to claim 4 wherein the disruption occurs by placing the cells in the superficial moist dermis and allowing the growing hair shafts to egress spontaneously.

8. A method according to claim 1 wherein the mammal is a mouse.

9. A method according to claim 1 wherein the mammal is a human.

10. A method according to claim 1 wherein the mixture of injected dermal cells and epidermal cells grow into an infundibular cyst, and hair follicles grow from the cyst.

11. A patch assay for assessing the hair follicle inductive property of disassociated mammal cells comprising the steps of:
a) providing a mixture of dissociated cells comprising dermal cells and epidermal cells;
b) injecting the mixture into a dermis/hypodermis of a mammal producing a dermal bleb; and
c) permitting the injected cellular mixture to grow a new hair follicle.

12. A method according to claim 11 wherein the assay is used to test the hair follicle inductive property of test materials.

13. A method according to claim 12 where in the test materials include pharmaceutical agents, chemical compounds, polymeric compounds, growth factors, cellular products, living cells, or biomolecules.

14. A method according to claim 11 wherein the ratio of dermal cells to epidermal cells falls in the range of about 100:1 to about 1:20.

15. A method according to claim 11 wherein the ratio of dermal cells to epidermal cells falls in the range of about 20:1 to about 1:2.

16. A method according to claim 11 further including the step of permitting the hair follicle to egress by disrupting the dermis/hypodermis adjacent to the hair follicle.

17. A method according to claim 11 wherein the disruption occurs by cutting the dermis/hypodermis.

18. A method according to claim 16 wherein disruption occurs by inserting a hollow wire tube through the dermis/epidermis and permitting the hair follicles to egress through the tube.

19. A method according to claim 11 wherein the mammal is a mouse.

20. A method according to claim 11 wherein the mammal is a human.

21. A method according to claim 11 wherein the mixture of injected dermal cells and epidermal cells grows into an infundibular cyst, and hair follicles grow from the cyst.
